(19) United States
(12) Patent Application Publication
(10) Pub. No.: US 2025/0099665 A1
(43) Pub. Date: Mar. 27, 2025

(54) FILTRATION WASHING OF HUMAN RED BLOOD CELLS

(71) Applicant: Ohio State Innovation Foundation, Columbus, OH (US)

(72) Inventors: Andre PALMER, Columbus, OH (US); Shuwei LU, Columbus, OH (US); Megan ALLYN, Columbus, OH (US)

(21) Appl. No.: 18/729,106

(22) PCT Filed: Nov. 15, 2022
(86) PCT No.: PCT/US2022/049988

§ 371 Date: Jul. 15, 2024

Related U.S. Application Data
(60) Provisional application No. 63/299,971, filed on Jan. 15, 2022.

(51) Int. Cl. A61M 1/34; A61M 1/02

(52) U.S. Cl. CPC A61M 1/3413; A61M 1/0281

(57) ABSTRACT

Methods for removing a contaminate from a blood product and preparing a regenerated blood product from an expired blood product, the methods comprising: filtering the blood product by filtration against a filtration membrane using a low-shear pump, thereby forming a retentate fraction comprising a washed blood product and a permeate fraction comprising the contaminate.

### CROSS REFERENCE TO RELATED APPLICATIONS

This application claims benefit of U.S. Provisional Application No. 63/299,971, filed Jan. 15, 2022, which is hereby incorporated by reference in its entirety.

### STATEMENT ACKNOWLEDGING GOVERNMENT SUPPORT

This invention was made with government support under award number R01HL126945, R01HL131720, R01HL138116, R01HL 156526, and R01EB021926 awarded by the National Institutes of Health. The government has certain rights in the invention.

### TECHNICAL FIELD

This application relates generally to systems and methodology for removing contaminates from a blood product.

### BACKGROUND

Red blood cells (RBCs) degrade during ex vivo storage, and lead to the accumulation of toxic hemolysis byproducts in the unit such as hemoglobin (Hb) during the maximum 42 day storage period set by the US FDA. Upon transfusion, cell-free Hb in the stored RBC unit can extravasate from the blood volume into the tissue space, where it scavenges nitric oxide (NO), a potent vasodilator, and elicits vasoconstriction and systemic hypertension within the patient. Additionally, tissue extravasation of cell-free Hb leads to tissue deposition of iron, and inevitably leads to oxidative tissue injury.

Therefore, in light of the accumulation of hemolysis byproducts during ex vivo RBC storage, RBC washing is often employed to remove accumulated waste products within an RBC unit prior to transfusion to mitigate any potential side-effects. Several commercially available technologies are clinically employed to wash stored RBC units prior to transfusion. For example, the ACP 215 cell processor and the COBE 2991 cell processor are commercially used devices for washing blood. Hansen A, Yi Q L, Acker J P. Quality of red blood cells washed using the ACP 215 cell processor: Assessment of optimal pre- and postwash storage times and conditions. Transfusion. 2013; 53(8):1772-9. Bennett-Guerrero E, Kirby B S, Zhu H, Herman A E, Bandarenko N, McMahon T J. Randomized study of washing 40- to 42-day-stored red blood cells. Transfusion. 2014; 54(10):2544-52.

Manual washing of single RBC units is an attractive approach due to its' low cost, but is laborious, limited in processing volume by the available centrifuge cup size, and exposes the unit to a high risk of bacterial contamination. In contrast, automated RBC unit washing systems are most commonly used in clinical settings to remove toxic byproducts. Some prior techniques use open cell processing system that utilizes centrifugation to facilitate separation based on differences in blood component density and can effectively reduce proinflammatory markers, restoring overall RBC quality near the end of the unit's ex vivo shelf life. Unfortunately, levels of hemolysis have been shown to rapidly increase after washing with these techniques, and often surpass prewashed levels before the 24 hour transfusion window is reached. Additional work investigating the ability of the these devices to wash 40 to 42 day stored RBC units showed that after washing, these techniques are unable to provide significant reduction in total cell-free Hb after the washing process, Hb being a toxic byproduct of the storage lesion. Regarding this limitation, it is clear that there is an urgent need for an innovative, easy to use RBC washing system that addresses the current pitfalls of both manual and automated washing systems.

### SUMMARY

In one aspect disclosed herein is a method directed to removing a contaminate from a blood product. Methods of removing the contaminate from the blood product can comprise filtering the blood product by filtration against a filtration membrane using a low-shear pump, thereby forming a retentate fraction comprising a washed blood product and a permeate fraction comprising the contaminate.

Also disclosed herein are methods directed to preparing a regenerated blood product from an expired blood product comprising filtering the expired blood product by filtration against a filtration membrane using a low-shear pump, thereby forming a retentate fraction comprising the regenerated blood product and a permeate fraction comprising a contaminate.

Further disclosed herein are systems for removing a contaminate from a blood product. These systems can comprise a blood product reservoir for receiving the blood product; and a filtration unit in fluid communication with the blood product reservoir. The filtration unit can comprise a filtration membrane; a conduit defining a path for recirculating fluid flow from the blood product reservoir to the filtration membrane and back to the blood product reservoir; and a low-shear pump operatively coupled to the fluid flow path of the blood product reservoir so as to direct the blood product along the path for recirculating fluid flow.

In some embodiments, the system can further comprise a was fluid reservoir containing a wash fluid and a conduit defining a path for one-way fluid flow from the wash fluid reservoir to the blood product reservoir. In some embodiments, the system can further comprise a waste product reservoir containing a contaminate and a conduit defining a path for one-way fluid flow from a permeate stream of the filtration membrane to the waste product reservoir containing the contaminate.

### DESCRIPTION OF DRAWINGS

FIG. 1 depicts a process flow diagram for an embodiment of the RBC washing process. 10 single RBC units were processed using the TFF RBC washing system. (1) Reservoir containing 0.9 wt % saline. (2) Sample port used for retentate sampling. (3) Retentate vessel, 0.65 μm TFF filter used to wash RBCs. (4) Centrifugal pump. (5) Permeate waste from the process (contains species <0.65 μm in size). (6) Cell waste. Arrows indicate the direction of flow.

FIGS. 2A-2C depict plots obtained from Example 1. Time per diacycle during the RBC washing process did not vary significantly between diacycles (p=0.999, NS) (A). HCT was standardized to 45% at the 0× diacycle and did not decrease significantly over the course of washing (p=0.124, NS) (B). Cell count within the TFF system was measured across diacycles (C). An ANOVA analysis for cell count change from the 0× diacycle to 10× diacycle were significant (p=0.0107, *), with additional significance found between the 0× diacycle to the 4× diacycle (p=0.031, *). 10 single RBC units were processed using the TFF RBC washing system.

FIGS. 3A-3B depict plots obtained from Example 1. Retentate cell-free Hb concentration (A) over 10 diacycles of RBC washing using TFF. 10 single RBC units were processed using the TFF RBC washing system. An ANOVA test was performed on the data subsets 0× to 10× diacycles, 0× to 4× diacycles, and 4× to 10× diacycles (α=0.05, Hₒ=Hb concentration is independent of the diacycle). Significance was found within the 0× to 10× diacycles (p=8E−16, ***), and 0× to 4× diacycles (p=2.8E−7, ***) subgroups. Significance was found between the 0× diacycle and 1×, 2×, 3×, and 4× diacycles using TukeyHSD (p=0.001, 2.9E−5, 1.6E−6, and 7E−7 respectively). No significance was found within the 4× to 10× diacycles subgroup (p=0.458, NS). Permeate cell-free Hb concentration (B) over 10 diacycles of RBC washing using TFF. An ANOVA test was performed on the data subsets 1× to 10× diacycles, 1× to 4× diacycles, and 4× to 10× diacycles (α=0.05, Hₒ=Hb concentration is independent of the diacycle). There was significance within the data for 1× to 10× diacycles (p=6.2E−15, ***), and for 1× to 4× diacycles (p=1.6E−5, ***). Within the 1× to 4× diacycle subgroup, significant differences were found between the 1× diacycle and 2×, 3×, 4× diacycles using TukeyHSD (p=0.006, 0.0001, and 3.2E−5 respectively). No significance was found within the 4× to 10× diacycle (p=0.151, NS).

FIGS. 4A-4C depict plots obtained from Example 1. Oxygen equilibrium curve (OEC) of both the initial RBC unit and 10× dicycle sample (A). 10 single RBC units were processed using the TFF RBC washing system. The OEC of the initial RBC unit is shown in blue with a dark grey 95% CI. The OEC of the final 10× sample is shown in red with a dark grey 95% CI. P₅₀ values (B) of the initial sample from the RBC unit and the final sample (10× diacycle) post TFF wash process (p=0.0493, *). Hill coefficient (n) (C) of the initial sample from the RBC unit and the final sample (10× diacycle) after the TFF wash process (p=0.0493, *).

FIG. 5 depicts a plot obtained from Example 2. Cell concentration was measured for unexpired RBC units during the TFF RBC washing process. An ANOVA analysis for cell concentration from the 0× diacycle to 10× diacycle did not find a significant change in the concentration of cells (p=0.46, NS). A total of 7 replicates were completed.

FIG. 6 depicts a plot obtained from Example 2. Oxygen equilibrium curve (OEC) of both the initial unexpired RBC unit and the 10× final diacycle sample. The OEC of the initial unexpired RBC unit is shown in blue with a dark grey 95% CI. The OEC of the final 10× sample is shown in red with a dark grey 95% CI. A total of 7 replicates were completed.

FIG. 7 depicts a plot obtained from Example 2. P₅₀ values of both the initial sample from the unexpired RBC unit and the final 10× sample were obtained from the OEC and no significance was found (p=0.39, NS). A total of 7 replicates were completed.

FIG. 8 depicts a plot obtained from Example 2. Hill coefficient of the initial unexpired RBCs from the RBC unit and post washing after 10× diacycles did not differ significantly (p=0.351, NS). A total of 7 replicates were completed.

FIG. 9 depicts a plot obtained from Example 2. HCT was standardized to 45% at the 0× diacycle and decreased significantly throughout the washing process (p=0.000133, ***). A total of 7 replicates were completed.

FIG. 10 depicts a plot obtained from Example 2. Time per diacycle was measured during the RBC washing process and did not vary significantly between each diacycle (p=0.944, NS).

FIG. 11 depicts a plot obtained from Example 2. Permeate cell-free Hb over 10× diacycles of RBC washing using TFF. An ANOVA test was performed on the data (p=5.76E−6, ***), as well as the data subsets 1× to 3× diacycles (p=0.0132, *) and 3× to 10× diacycles (p=0.278, NS). A total of 7 replicates were performed.

FIG. 12 depicts a plot obtained from Example 2. Retentate cell-free Hb concentration over 10× diacycles of RBC washing using TFF. An ANOVA test was performed on the data (p=6.9E−11, ***), as well as the subsets from 0× to 2× diacycles (p=0.00329, **), and 2× to 10× diacycles (p=0.748, NS). A total of 7 replicates were performed.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims, which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example embodiments.

As used herein, the term “substantially” means that the subsequently described event or circumstance completely occurs or that the subsequently described event or circumstance generally, typically, or approximately occurs.

As used herein, the term “substantially,” in, for example, the context “substantially identical” or “substantially similar” refers to a method or a system, or a component that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

It is understood that in aspects disclosed herein, the terms “diacycle” and “diafiltration cycles” are used interchangeably.

As used herein, the term “tangential-flow filtration” refers to a process in which the fluid mixture containing the components to be separated by filtration is recirculated at velocities tangential to the plane of the filtration membrane to reduce fouling of the filter. In such filtrations a pressure differential is applied along the length of the filtration membrane to cause the fluid and filterable solutes to flow through the membrane (i.e. filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

The present invention may be understood more readily by reference to the following detailed description of various aspects of the invention and the examples included therein and to the Figures and their previous and following description.

### Methods

Disclosed herein are methods directed to removing a contaminate from a blood product. Methods of removing the contaminate from the blood product can comprise filtering the blood product by filtration against a filtration membrane using a low-shear pump, thereby forming a retentate fraction comprising a washed blood product and a permeate fraction comprising the contaminate. Additionally disclosed herein are methods directed to preparing a regenerated blood product from an expired blood product comprising filtering the expired blood product by filtration against a filtration membrane using a low-shear pump, thereby forming a retentate fraction comprising the regenerated blood product and a permeate fraction comprising a contaminate.

In some embodiments, filtering the blood product (or the expired blood product) can further comprise washing the blood product (or the expired blood product by filtration against a filtration membrane using a low-shear pump. For example, this can comprise adding additional volumes of a wash buffer to the system followed by additional filtration so as to remove contaminants from the blood product (or expired blood product).

Various embodiments of blood products can be filtered and washed using the methods disclosed herein. For example, the blood product may be whole blood, white blood cells, red blood cells, platelets, blood plasma and blood plasma proteins. In particular aspects, the blood product may be packed red blood cells for blood transfusions. The present method can be used to reduce the concentration of a contaminate from expired and/or unexpired blood products to prolong storage. An expired blood product may refer to blood that has exceeded its storage period recommendations according to FDA Guidelines, which is hereby incorporated by reference. Food and Drug Administration (FDA). CFR—Code of Federal Regulations Title 21. Vol. 21, Www.Fda.Gov. 2019. For example, expired packed red blood cells may include to blood products that have exceeded its 42 day storage period recommendations. In some embodiments, the expired blood product can be a blood product that is at least 30 days from the date of collection, such as at least 35 days from the date of collection, at least 40 days from the date of collection, at least 45 days from the date of collection, at least 50 days from the date of collection, at least 55 days from the date of collection, at least 60 days from the date of collection, at least 65 days from the date of collection, at least 70 days from the date of collection, or at least 75 days from the date of collection.

The methods disclosed herein effectively reduce the concentration of one or more contaminates from a blood product. In some embodiments, the contaminate comprise a byproduct of hemolysis that can disrupt or change the viability of the blood product. For example, the contaminate may comprise extracellular proteins, such as hemoglobin (Hb), extracellular vesicles, heme, iron, cytokines, potassium ions, lactate, protons, and/or other cellular waste or debris. In particular aspects, the contaminate comprises cell-free hemoglobin. In various embodiments, the methods disclosed herein can be used to remove multiple contaminates from the blood product. For example, the method may be used to remove cell-free Hb, heme, iron, potassium, lactate, protons, and/or other cellular waste or debris, or combinations thereof.

The membranes useful in the filtration and washing steps described herein can be in the form of flat sheets, rolled-up sheets, cylinders, concentric cylinders, ducts of various cross-section and other configurations, assembled singly or in groups, and connected in series or in parallel within the filtration unit. The unit can be constructed so that the filtering and filtrate chambers run the length of the membrane.

Suitable membranes include those that separate the desired species from undesirable species in the mixture without substantial clogging problems and at a rate sufficient for continuous operation of the system. Examples are described, for example, in Gabler F R. Tangential flow filtration for processing cells, proteins, and other biological components. ASM News 1984; 50:299-304.

Generally, the filtration membrane can comprise a filtration membrane. Filtration membranes are normally asymmetrical with a thin film or skin on the upstream surface that is responsible for their separating power. They are commonly made of regenerated cellulose, polysulfone or polyethersulfone. In some cases, filtration membrane can be rated for removing solutes having a molecular weight or molecular diameter of from 1 g/mol to 30 μm, such as from 1 g/mol to 25 μm, from 1 g/mol to 20 μm, from 1 g/mol to 15 μm, from 1 g/mol to 10 μm, from 1 g/mol to 5 μm, from 1 g/mol to 4 μm, from 1 g/mol to 3 μm, from 1 g/mol to 2 μm, from 1 g/mol to 1 μm, from 1 g/mol to 0.65 μm, from 1 g/mol to 0.2 μm, from 1 g/mol to 0.1 μm, from 1 g/mol to 50 nm, from 1 g/mol to 750 kDa, from 1 g/mol to 500 kDa, from 1 g/mol to 300 kDa, from 1 g/mol to 100 kDa, from 1 g/mol to 70 kDa, from 1 g/mol to 50 kDa, from 1 g/mol to 30 kDa, from 1 g/mol to 10 kDa, from 1 g/mol to 5 kDa, from 1 g/mol to 3 kDa, or from 1 g/mol to 1 kDa.

The methods described herein can employ direct-flow filtration (DFF), cross-flow or tangential-flow filtration (TFF), or a combination thereof. In certain embodiments, the methods described herein can employ TFF. One example of a suitable hollow fiber filter module is the MiniKros Sampler obtained from Repligen (S02-E65U-07N).

In certain aspects, the filtration comprises constant-volume filtration. During constant-volume filtration, the amount of a wash solution flowing into the filtration system is substantially equal to the amount of fluid leaving through the permeate. This is generally accomplished by pumping replacement solution to the feed tank so as to keep the fluid level fixed.

In some cases, each filtration step can involve filtration through a single filtration membrane. In other cases, because membrane filters are not perfect and may have holes that allow some intended retentate molecules to slip through, more than one membrane (e.g., two membranes, three membranes, four membranes, or more) having the same pore size can be utilized for a given filtration step. In these embodiments, the membranes can be placed so as to be layered parallel to each other (e.g., one on top of the other) such that filtered fluid sequentially flows through each of the more than one membrane. Membrane filters for tangential-flow filtration are available as units of different configurations depending on the volumes of liquid to be handled, and in a variety of pore sizes.

The filtration unit useful herein is suitably any unit now known or discovered in the future that serves as an appropriate filtration module, particularly for filtration. The preferred filtration unit is hollow fibers or a flat sheet device. These sandwiched filtration units can be stacked to form a composite cell. One example type of rectangular filtration plate type cell is available from Filtron Technology Corporation, Northborough, Mass., under the trade name Centrasette. Another example filtration unit is the Millipore Pellicon filtration system available from Millipore, Bedford, Mass.

Generally, a diafiltration cycles is defined by the circulation of a volume of fluid within the filtration system. In some cases, multiple diafiltration cycles are used to remove a contaminate from the blood product. For example, some aspects of the present method may include filtering the blood product for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles. In some aspects, the blood product is filtered at a hypothermic temperature. For example, the blood may be filtered at a temperature less than 37° C., such as less than 30° C., less than 25° C., less than 20° C., less than 15° C., less than 10° C., less than 5° C., less than 0° C.

The methods described herein include a low-shear pump to circulate the blood product. Low-shear pumps maintain flow rates that limits the amount of turbulent flow through the system and thereby create less shear stress on the blood product compared to other pumps. Generally, the blood product contacts the filter membrane by a pumping system, which passes the blood product through the lumen side of the hollow fiber. In particular embodiments, the low shear pump is a biocompatible pump. Examples of pumping systems include peristaltic pumps, double diaphragm pumps, centrifugal pumps (PuraLev i30SU, Levitronix®), and other low-shear bioprocessing pumps (Levitronix® pumps, Zurich, Switzerland) and alternating tangential flow systems (ATF™, Refine Technology, Pine Brook, N.J., See e.g. U.S. Pat. No. 6,544,424; Furey (2002) Gen. Eng. News. 22 (7), 62-63.). The permeate may be drawn from the filters by use of, for example, low-shear peristaltic pumps. In some aspects, the pump is selected and configured such that the inner wall of the filtration membrane in contact with the blood product is subjected to a shear rate less than 5,000 s⁻¹, such as less than 4,000 s⁻¹, less than 3,000 s⁻¹, less than 2,000 s⁻¹, less than 1,000 s⁻¹, or less than 500 s⁻¹.

In various aspects, the contaminate is present at a first concentration in the blood product and present in a second concentration in the washed blood product, wherein the second concentration in lower than the first concentration. In some embodiments, the second concentration is 50% or less of the first concentration, such as 40% or less of the first concentration, 30% or less of the first concentration 20% or less of the first concentration, 10% or less of the first concentration, 5% or less of the first concentration, 2.5% or less of the first concentration, or 1% or less of the first concentration. In various aspects, the second concentration is 0.1 mM or less, such as 0.05 mM or less, 0.04 mM or less, 0.03 mM or less, 0.02 mM or less, or 0.01 mM or less of the contaminate.

In some embodiments, the second concentration is 50% or less of the first concentration after the blood product is filtered for 4 diafiltration cycles, such as at least 40% or less of the first concentration, 30% or less of the first concentration 20% or less of the first concentration, 10% or less of the first concentration, 5% or less of the first concentration, 2.5% or less of the first concentration, or 1% or less of the first concentration after 4 diafiltration cycles. In some aspects, the second concentration is 50% or less of the first concentration after the blood product is filtered for 3 diafiltration cycles, such as at least 40% or less of the first concentration, 30% or less of the first concentration 20% or less of the first concentration, 10% or less of the first concentration, 5% or less of the first concentration, 2.5% or less of the first concentration, or 1% or less of the first concentration after 3 diafiltration cycles. Similarly in some embodiments, the second concentration is 50% or less of the first concentration after the blood product is filtered for 2 diafiltration cycles, such as at least 40% or less of the first concentration, 30% or less of the first concentration 20% or less of the first concentration, 10% or less of the first concentration, 5% or less of the first concentration, 2.5% or less of the first concentration, or 1% or less of the first concentration after 2 diafiltration cycles.

### Systems

Further disclosed herein are systems for removing a contaminate from a blood product. These systems can comprise a blood product reservoir for receiving the blood product; and a filtration unit in fluid communication with the blood product reservoir. The filtration unit can comprise a filtration membrane; a conduit defining a path for recirculating fluid flow from the blood product reservoir to the filtration membrane and back to the blood product reservoir; and a low-shear pump operatively coupled to the fluid flow path of the blood product reservoir so as to direct the blood product along the path for recirculating fluid flow.

In some embodiments, the system can further comprise a was fluid reservoir containing a wash fluid and a conduit defining a path for one-way fluid flow from the wash fluid reservoir to the blood product reservoir. In some embodiments, the system can further comprise a waste product reservoir containing a contaminate and a conduit defining a path for one-way fluid flow from a permeate stream of the filtration membrane to the waste product reservoir containing the contaminate.

Various embodiments of blood products can be used in the systems disclosed herein. For example, the blood products may be whole blood, white blood cells, red blood cells, platelets, blood plasma and blood plasma proteins. In some examples, the blood product may be packed red blood cells for blood transfusions. The present system can be used to reduce the concentration of a contaminate from expired and/or unexpired blood products to prolong storage. In some embodiments, the expired blood product comprises a blood product that is at least 45 days from the date of collection, such as at least 50 days from the date of collection, at least 55 days from the date of collection, at least 60 days from the date of collection, at least 65 days from the date of collection, at least 70 days from the date of collection, at least 75 days from the date of collection, at least 80 days from the date of collection, at least 85 days from the date of collection, or at least 90 days from the date of collection.

The systems disclosed herein can effectively reduce the concentration of one or more contaminates from a blood product. In some aspects, the contaminate comprise a byproduct of hemolysis that can disrupt or change the viability of the blood product. For example, the contaminate may comprise cell-free Hb, heme, iron, potassium, lactate, protons, and/or other cellular waste or debris. In particular aspects, the contaminate comprises cell-free hemoglobin. In certain other embodiments, the methods disclosed herein can be used to remove multiple contaminates from the blood product. For example, the method may be used to remove cell-free Hb, heme, iron, potassium, lactate, protons, and/or other cellular waste or debris, or combinations thereof. In some embodiments, the contaminate can comprise an extracellular protein, such as hemoglobin (Hb), extracellular vesicles, cytokines, heme, iron, potassium ions, lactate, protons, or any combination thereof. The reservoir for receiving the blood product may be various sizes to accommodate different volumes of the blood product. For example, the reservoir may have a volume between 100 mL to 5 L, such as from 100 mL to 3 L, from 100 mL to 1 L, from 100 mL to 500 mL, or from 200 mL to 350 mL. In other aspects, the volume may comprise a larger volume for receiving a larger sample of blood products for washing. For example, the reservoir may have a volume greater than 5 L, such as greater than 10 L, greater than 15 L, greater than 25 L, or greater than 30 L. The blood product reservoir for receiving the blood product can be in fluid communication with the filtration membrane such to create a conduit defining a path for recirculating fluid. In certain embodiments, a low-shear pump is operatively coupled at a location in the fluid flow path to circulate the fluid within the recirculating fluid flow path.

The filtration membrane of the present system can comprise an filtration membrane. In some aspects, the filtration membrane comprises regenerated cellulose, polysulfone or polyethersulfone. In some cases, the filtration membrane can be rated for removing solutes having a molecular weight or molecular diameter of from 1 g/mol to 30 μm, such as from 1 g/mol to 25 μm, from 1 g/mol to 20 μm, from 1 g/mol to 15 μm, from 1 g/mol to 10 μm, from 1 g/mol to 5 μm, from 1 g/mol to 4 μm, from 1 g/mol to 3 μm, from 1 g/mol to 2 μm, from 1 g/mol to 1 μm, from 1 g/mol to 0.65 μm, from 1 g/mol to 0.2 μm, from 1 g/mol to 0.1 μm, from 1 g/mol to 50 nm, from 1 g/mol to 750 kDa, from 1 g/mol to 500 kDa, from 1 g/mol to 300 kDa, from 1 g/mol to 100 kDa, from 1 g/mol to 70 kDa, from 1 g/mol to 50 kDa, from 1 g/mol to 30 kDa, from 1 g/mol to 10 kDa, from 1 g/mol to 5 kDa, from 1 g/mol to 3 kDa, or from 1 g/mol to 1 kDa. The systems described herein can employ direct-flow filtration (DFF), cross-flow or tangential-flow filtration (TFF), or a combination thereof. In certain aspects, the systems described herein can employ TFF.

In some embodiments, the filtration can include multiple filtration membranes (e.g., two membranes, three membranes, four membranes, or more) having the same or different pore size. In these embodiments, the membranes can be placed so as to be layered parallel to each other (e.g., one on top of the other) such that filtered fluid sequentially flows through each of the more than one membrane.

By way of non-limiting illustration, examples of certain aspects of the present disclosure are given below.

### EXAMPLES

The following examples are put forth so as to provide those of ordinary skill in the art with a complete disclosure and description of how the compounds, compositions, articles, devices, and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, the temperature is in degrees C. or is at ambient temperature, and pressure is at or near atmospheric.

### Example 1

### Introduction

Red blood cells (RBCs) degrade during ex vivo storage, and lead to the accumulation of toxic hemolysis byproducts in the unit such as hemoglobin (Hb) during the maximum 42 day storage period set by the US FDA.[1][2] Upon transfusion, cell-free Hb in the stored RBC unit can extravasate from the blood volume into the tissue space, where it scavenges nitric oxide (NO), a potent vasodilator, and elicits vasoconstriction and systemic hypertension within the patient.[3] Additionally, tissue extravasation of cell-free Hb leads to tissue deposition of iron, and inevitably leads to oxidative tissue injury.[4]

Therefore, in light of the accumulation of hemolysis byproducts during ex vivo RBC storage, RBC washing is often employed to remove accumulated waste products within an RBC unit prior to transfusion to mitigate any potential side-effects.[5,6] Several commercially available technologies are clinically employed to wash stored RBC units prior to transfusion.[7,8]

Manual washing of single RBC units is an attractive approach due to its' low cost, but is laborious, limited in processing volume by the available centrifuge cup size, and exposes the unit to a high risk of bacterial contamination.[5,9] In contrast, automated RBC unit washing systems are most commonly used in clinical settings to remove toxic byproducts, one example is the COBE 2991 cell processor (Terumo, Somerset, NJ).[7,10] The COBE 2991 is an open cell processing system that utilizes centrifugation to facilitate separation based on differences in blood component density and can effectively reduce proinflammatory markers, restoring overall RBC quality near the end of the unit's ex vivo shelf life.[9,10] Unfortunately, levels of hemolysis have been shown to rapidly increase after washing with the COBE 2991, and often surpass prewashed levels before the 24 hr transfusion window is reached.[9] Additional work investigating the ability of the COBE 2991 to wash 40 to 42 day stored RBC units showed that after washing, the COBE 2991 is unable to provide significant reduction in total cell-free Hb after the washing process, Hb being a toxic byproduct of the storage lesion.[11] Regarding this limitation, it is clear that there is an urgent need for an innovative, easy to use RBC washing system that addresses the current pitfalls of both manual and automated washing systems.

Considering the plethora of centrifugation-based RBC washing systems in existence, there has been substantially less research into the use of tangential flow filtration (TFF) for RBC unit washing, with no commercially available system on the market. TFF utilizes a porous hollow fiber or flat sheet membrane to enable continuous flow purification. Molecules larger than the pore size cutoff of the membrane are retained on the membrane and in the system, while molecules smaller than the pore size cutoff permeate through the membrane and are removed from the system. The use of TFF techniques on whole blood currently utilize gravity-driven separation, and are primarily focused on the separation of whole blood into plasma and RBC fractions.[12,13] Compared to centrifugal separation, TFF systems can process a wide range of RBC concentrate volumes, allows for easy storage solution exchange, and has the ability to maintain sterility via the use of autoclavable materials and a closed loop system. Additionally, the currently designed TFF system is light weight and easily transportable, with the system as a whole (system vessel, pump, tubing, and hollow fiber filter) weighing less than 2 kg. One study used TFF to wash RBCs in diafiltration mode, and concentrated a cryopreserved RBC unit, but resulted in significant RBC lysis (likely due to use of a high shear peristaltic pump), and focused on investigating the rheological properties of the washed unit.[14] Alternatively, in this example we explored implementation of a novel TFF system using a low shear stress inducing centrifugal pump to separate stored RBCs from their primary hemolysis byproduct Hb. This example developed a system that effectively washes RBC units as demonstrated by the successful removal of cell-free Hb and shows negligible process-induced hemolysis, providing a viable alternative to current manual and automated RBC washing systems.

### Materials & Methods

Materials. Sodium chloride (NaCl), sodium hydroxide (NaOH), and 0.2 μm Titan3 sterile filters were purchased from Fisher Scientific (Waltham, MA). Hollow fiber TFF modules (S02-E65U-07N, modified polyethersulfone membrane, 0.65 μm pore size, composed of 110 individual hollow fibers, 0.75 mm internal diameter, 520 cm² total surface area) were purchased from Repligen (Rancho Dominguez, CA). A biocompatible centrifugal pump (PuraLev i30SU) that exposes cells to low shear stresses was purchased from Levitronix (Framingham, MA). A minicentrifuge (50-090-100, working speed 6,000 rpm, max speed 6,600 rpm) from Fisher Scientific (Waltham, MA) was used to separate RBCs from the wash solution. Expired leuko-reduced packed human RBCs (RBC units, 60-70 days old, stored in AS-1) were generously donated by the Transfusion Services of the Wexner Medical Center at The Ohio State University, Columbus, Ohio. The RBC units used in this example were expired and deidentified and thus required no Ethics Committee approval.

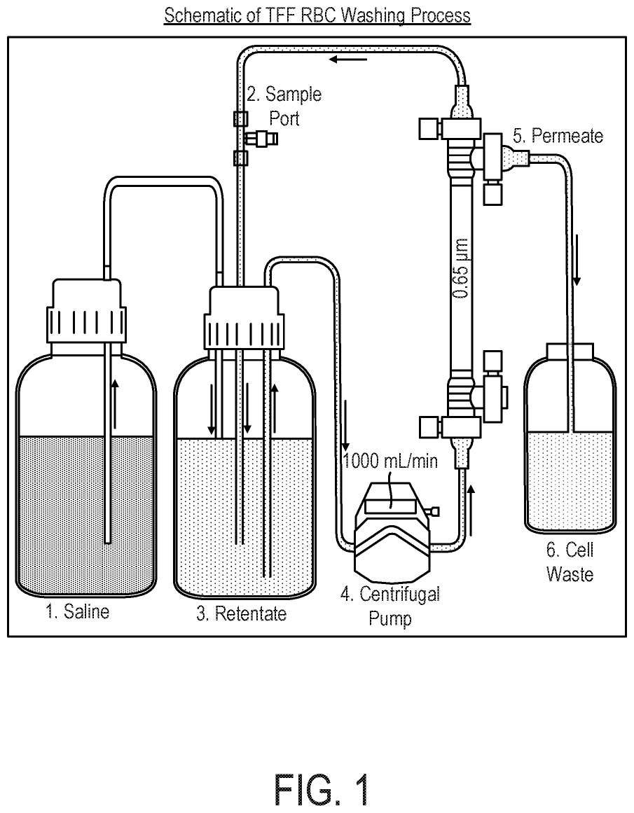

RBC Washing. The TFF-facilitated RBC washing process was performed on individual stored RBC units expired past the FDA regulated 42-day storage period. All RBC units were stored and washed at 4° C. in a chromatography refrigerator. A single RBC unit was transferred to a 1 L Nalgene container by opening and transferring the RBC unit in a sterile biosafety cabinet. The hematocrit (HCT) in the total system volume (which includes the combined fluid volume in the TFF filter, lines, and retentate vessel) was standardized to 45% with 0.9 wt % saline. Prior to washing, single RBC units were mixed by gentle inversion to yield a homogenous cell suspension. An initial sample of the RBC unit was taken to establish baseline conditions prior to washing. FIG. 1 shows the general schematic of the TFF-facilitated RBC washing system. 0.9 wt % saline solution was diafiltered into the retentate vessel to maintain a constant system volume. The sample port in the RBC retentate loop was used to take retentate samples. The retentate line was connected to a centrifugal pump from the reservoir, which operated at a constant flow rate of 1000 ml/min and directed RBCs through the bottom of the TFF filter against gravity, with RBCs being retained in the retentate, while cell debris, proteins and other molecules smaller than 0.65 μm passing into the permeate. The permeate line enters a cell waste container with samples collected directly from the permeate line.

RBCs in the retentate vessel were first acclimated to the system components via circulation for 2 minutes with the permeate line closed. This ensured proper mixing of the RBCs in the system before starting the constant volume diafiltration cell washing process. During the acclimatization period, an initial 0× diacycle sample was taken to confirm the HCT of 45% was successfully achieved before initiating the diafiltration process. The total system volume was used to determine the volume per diacycle (i.e. one complete system exchange volume) and was measured by collecting permeate leaving the system. Retentate and permeate samples were taken at the end of each diacycle and stored at 4° C. for analysis. RBC units were washed with standard 0.9 wt % saline washing solution for the entirety of the process and were not stored ex vivo after the washing process was completed. Instead, newly washed RBCs were utilized for hemoglobin purification based on published procedure. A total of ten diacycles were completed per RBC wash for each individual RBC unit, with a total of ten individual RBC units being subjected to the TFF RBC washing process.

Hematocrit Analysis. The HCT was determined by injecting 65 μL of each retentate sample, including an initial sample from the RBC unit, into a mylar wrapped 75 mm capillary tube (Drummond, Broomall, PA) followed by centrifugation in a Sorvall Legend micro 17 microcentrifuge (Fisher Scientific, Waltham, MA) for 5 minutes to pellet the RBCs. Post centrifugation, the capillary tubes were quantified using a standardized HCT graph to obtain the HCT of RBCs in the retentate.

Total Hb Quantification. The cell-free Hb concentration was quantified via UV-visible absorbance spectrometry on a diode array spectrophotometer HP 8452A (Olis, Bogart, GA). Retentate supernatants were isolated via centrifugation using a minicentrifuge (Fisher Scientific, Waltham, MA) at 6000 RPM for 2 minutes to pellet the RBCs and analyzed after separation. Processed retentate and permeate samples were sterile filtered through a 0.2 μm Titan3 filter (Fisher Scientific, Waltham, MA) for UV-visible spectral analysis. Sterile filtration was employed to reduce light scattering during optical measurements to only quantify cell-free Hb. The Winterbourn equations were used to determine the total concentration of cell-free Hb in the permeate and retentate samples and further used for the cell-free Hb mass balance.[15] Quantification using UV-visible spectral analysis examines the absorbance of the various Hb oxidation species that could be present in a sample. Using the characteristic absorbance peaks: oxyhemoglobin at 577 nm, methemoglobin at 630 nm, and hemichrome at 560 nm and the defined extinction coefficients of each species at each wavelength previously described by Winterbourn, the quantity of each different species can be found and used to find the total Hb of the sample. The equations used for each species quantification are found below.

$$[ Oxyhemoglobin ] = {119}_{A 577} - {39}_{A 630} - {89}_{A 560} \qquad ( 1 )$$

$$[ Methemoglobin ] = {28}_{A 577} + {307}_{A 630} - {55}_{A 560} \qquad ( 2 )$$

$$[ Hemichrome ] = - {133}_{A 577} - {114}_{A 630} + {233}_{A 560} \qquad ( 3 )$$

Oxygen Equilibrium of RBCs. Oxygen equilibrium curves (OEC) for RBCs pre and post wash were measured using a Hemox Analyzer (TCS Scientific Corp., New Hope, PA) operated at 37±0.1° C. RBC samples were diluted into 5 mL of Hemox buffer (pH 7.4) with 20 μL additive A, 20 μL additive B, and 20 μL anti-foaming agent (TCS Scientific). Data obtained from the Hemox Analyzer was fit to the Hill equation using an Igor (Wavemetrics, Portland, OR) script to regress the oxygen affinity (P₅₀, pO₂ at which half of the Hb is saturated with oxygen), and Hill coefficient (n, cooperativity of 02 binding to Hb).[16]

RBC Viscosity. A Brookfield DV3T rheometer with a CP-40 spindle (Brookfield, Middleboro, MA) was used to measure the viscosity of RBC samples at 37° C. and a shear rate of 160 s⁻¹.[17,18]

RBC Cell Count. Cell counts for retentate samples were measured using a Multisizer 4e Coulter Counter (Beckman Life Sciences, Indianapolis, IN). RBC samples were diluted 100× prior to addition of 100 μL of the diluted cells into 20 mL of filtered Isoton solution (Beckman Life Sciences) prior to Coulter Counter analysis.

Data Analysis. Results are reported as the mean±standard deviation. RStudio (version 1.3.1093, RStudio Inc., Boston, MA) was used to analyze all data. A one-way ANOVA was utilized along with TukeyHSD posttest for data analysis. T-tests were used for P₅₀ and n initial and final comparisons. A two-tailed p-value<0.05 was considered statistically significant.

### Results

### Time for Each Diacycle Remained Constant

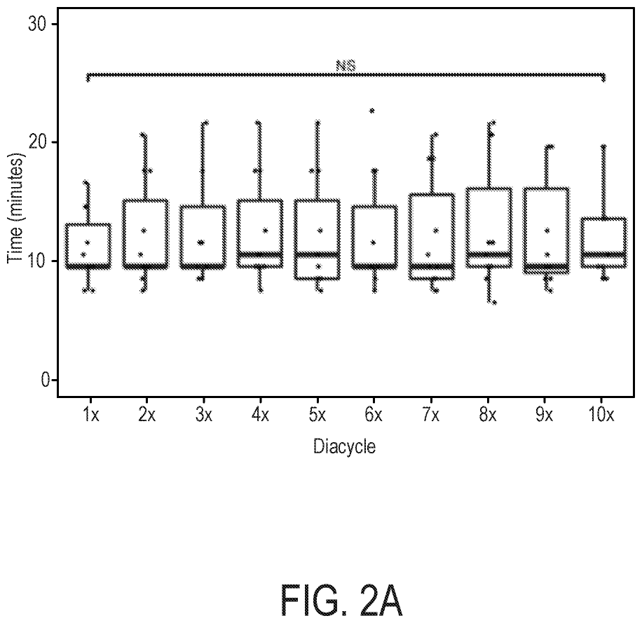

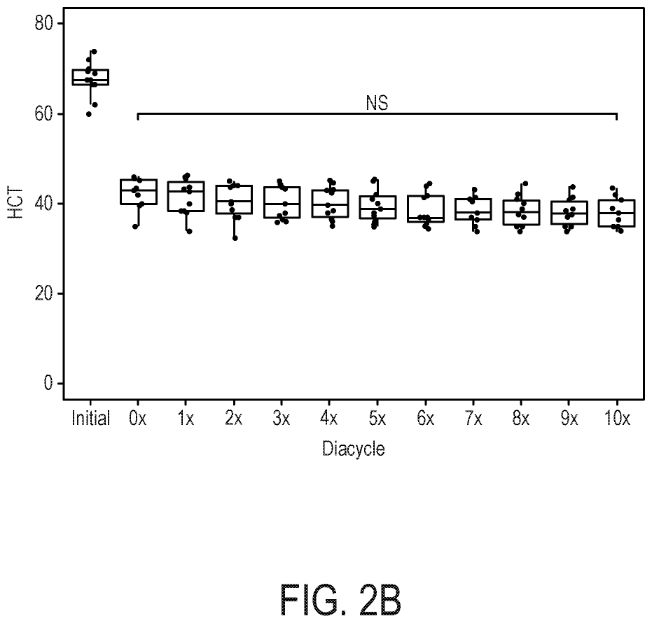

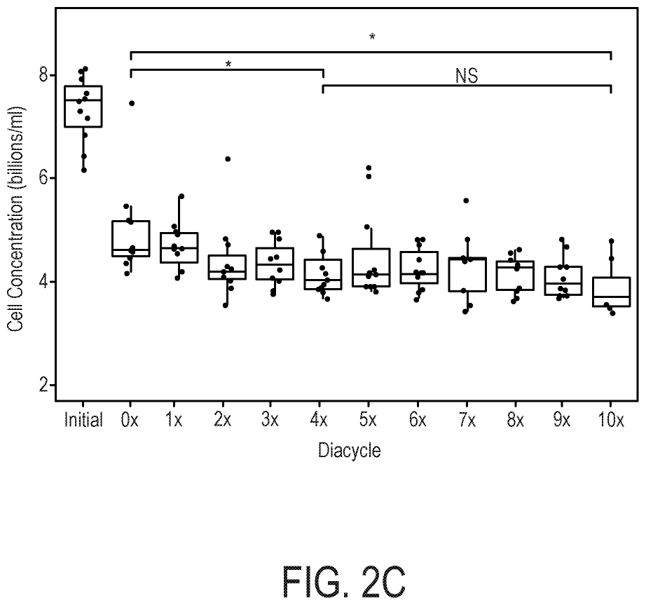

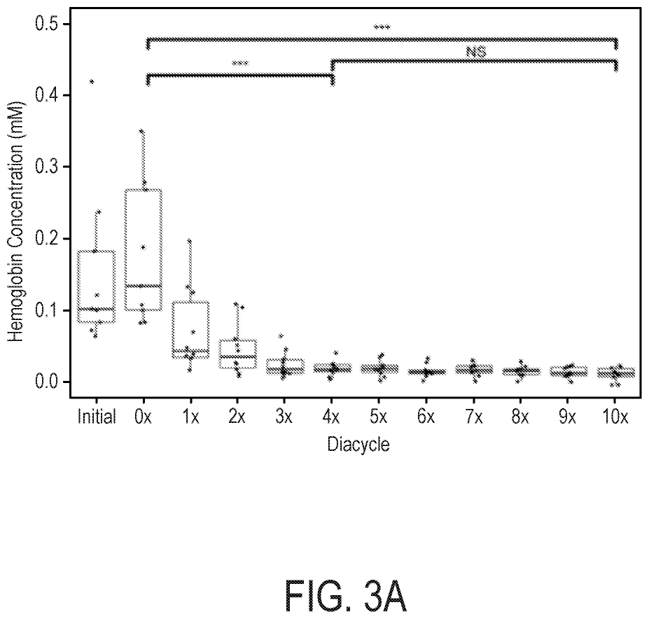

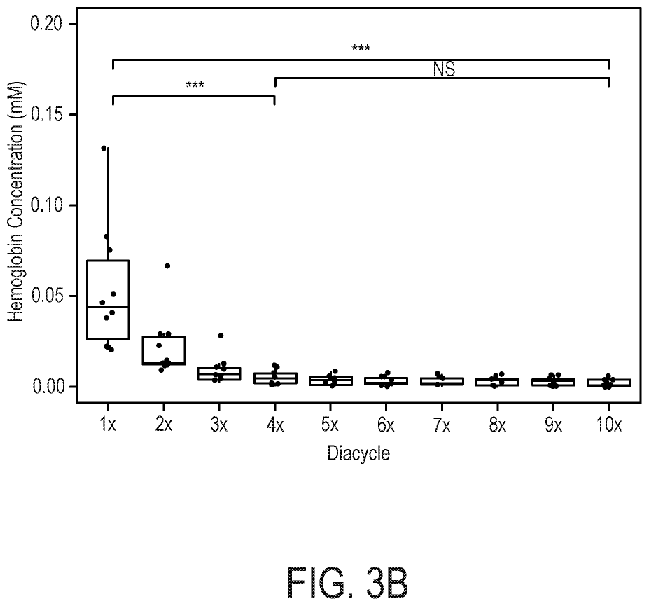

The average time per diacycle remained constant at ˜10 minutes throughout the RBC washing process for a total of 10 diacycles (FIG. 2A). The entire process takes 100 minutes to wash one RBC unit (10 diacycles) or 40 minutes to remove the majority of cell-free Hb (4 diacycles). The lesser washing time (40 minutes) necessary to remove the majority of cell-free Hb in the unit is triple the average 14 minutes washing time for processing a single unexpired RBC unit using the COBE 2991.[8] There was no significant difference in the time per diacycle during the washing process (p=0.999, NS). The residence time of RBCs in the retentate reservoir varied slightly due to the variance in the volume of each RBC unit, but on average, the system volume was ˜350 ml. Based on the system volume and the pump volumetric flow rate, the residence time was calculated to be 0.4 min (i.e. time for the system volume to complete one circuit in the TFF system).

### Hematocrit Remains Constant Throughout TFF Processing

RBCs from a single RBC unit (initial) were standardized to 45% HCT (0×) in the system from an initial HCT of ˜65% (FIG. 2B). The effect of each diacycle on the HCT in the system was analyzed using a one-way ANOVA from 0× to 10× diacycle. The HCT did not change significantly throughout the course of the RBC washing process (p=0.124, NS).

### RBC Concentration Remains Constant Across Diacycles

The concentration of RBCs in the retentate vessel was measured throughout the RBC washing process (FIG. 2C). The RBCs were measured at a diameter of 4.4 μm, which corresponds to the approximate spherical diameter of RBCs measured via Coulter Counter analysis. The initial RBC concentration is significantly higher than the 0× diacycle, due to standardization to 45% HCT. The initial RBC concentration measured directly from RBC units was ˜7.510±0.37 billion cells/ml and decreased to 4.625±0.35 billion cells/ml at the 0× diacycle after standardizing the HCT to 45%. These RBC concentration values are similar to values in the literature.[19] RBC concentration differences between diacycles were analyzed using one-way ANOVA comparing the entire RBC wash process from 0× to 10× diacycle and were found to show a significant decrease in cell concentration over the entire TFF process (p=0.0107, *). The RBC concentration decreased significantly from 4.625±0.35 billion cells/ml at 0× to 4.030±0.27 billion cells/ml at the 4× diacycle (p=0.031, *) corresponding to 87% cell recovery at the end of the wash process, with no significant cell loss after the 4× diacycle (p=0.356, NS). This suggests hemolysis of cells with significantly compromised cell membranes occurs early in the TFF washing process, and then tapers off as the process continues.

### Hb Concentration in the Retentate and Permeate Decreases Over Multiple Washing Diacycles

The retentate cell-free Hb concentration is shown. There is, on average, 0.105 mM of cell-free Hb within the RBC unit before processing. Post wash, the cell-free Hb concentration decreases to ˜0.0157 mM (at the end of the 10× diacycle). The TFF RBC washing process significantly reduces the cell-free Hb concentration after 4 diacycles (p=2.8E−7, ***) and remains constant from 4× to 10× (p=0.458, NS) diacycles. A Tukey honestly significant difference (HSD) test was performed within the 0× to 4× diacycles and found significance between the 0× to 1×, 2×, 3×, and 4× diacycles (p=0.001, 2.9E−5, 1.6E−6, 7E−7 respectively). Without being bound by theory, this suggests that the majority of cell-free Hb was removed at the beginning of the TFF wash process.

The permeate cell-free Hb concentration is shown. Significance was found within the 1× to 10× diacycle dataset (p=6.2E−15, ***). The overall dataset was then split into two subsets from 1× to 4× diacycle and from 4× to 10× diacycle with significance found within the 1× to 4× diacycle dataset (p=1.6E−5, ***). A Tukey HSD test was performed within the dataset from the 1× to 4× diacycle, and found that there was a significant difference between the 1× diacycle and the 2, 3×, and 4× diacycles (p=0.006, 0.0001, and 3.2E−5, respectively). No significance was found between the other diacycles. The US FDA considers stored RBC units with a hemolysis level less than 1% to be safe for transfusion. 1% hemolysis is roughly equivalent to a cell-free Hb concentration of 0.01 mM in the unit, which is lower than the final cell-free Hb concentration achieved in this example of ˜0.0157 mM observed post wash.[20] One must, however, remember that the RBC units in this example were outdated (60-70 days old), which likely contributed to the higher final cell-free Hb concentration compared to literature values for washing non-expired RBC units.[21]

### Total Cell-Free Hb Mass Balance

The total cell-free Hb for each diacycle was quantified in order to perform an overall cell-free Hb mass balance. The mass of cell-free Hb for retentate and permeate samples was averaged for all 10 replicates (Table 1). The initial mass of cell-free Hb in individual RBC units is on average, 2.06 g with a Hb concentration of 0.105 mM, which corresponds to a hemolysis level of ˜10%. After the completion of the first diacycle, the cell-free Hb in the retentate is ˜1.05 g, indicating that ˜50% of the extracellular Hb has been removed at this stage. Cell-free Hb continues to be removed from the retentate for all subsequent diacycles. The system reached a hemolysis level of 1±0.3%, which remains constant through the remaining 6 diacycles. Without wishing to be bound by theory, this suggests that TFF is effective at removing cell-free Hb after 4 diacycles.

Additionally, a cell-free Hb mass balance analysis on the permeate samples show significant Hb removal at the start of the diafiltration process (Table 1). The 1× diacycle is the first diacycle with permeate flow, and removes the majority of cell-free Hb. The total mass of cell-free Hb continually decreases in the permeate as washing proceeds, supporting the theory that the TFF system is not inducing additional shear stress on the RBCs to cause lysis beyond what is needed to enable separation of cell-free Hb from the remaining RBCs in the retentate.

TABLE 1
Cell-free Hb Overall Mass Balance.

| | |
|---|---|
| Retentate Cell-Free Hb | Permeate Cell-free Hb |

| Diacycle | Free Hb (g) | Free Hb (g) |
|---|---|---|
| Initial | 2.060 ± 1.539 | |
| 0X | 2.196 ± 1.191 | |
| 1X | 1.052 ± 0.834 | 1.648 ± 1.100 |
| 2X | 0.618 ± 0.347 | 0.678 ± 0.547 |
| 3X | 0.363 ± 0.187 | 0.273 ± 0.227 |
| 4X | 0.296 ± 0.106 | 0.173 ± 0.130 |
| 5X | 0.291 ± 0.106 | 0.114 ± 0.089 |
| 6X | 0.245 ± 0.084 | 0.097 ± 0.081 |
| 7X | 0.256 ± 0.070 | 0.100 ± 0.071 |
| 8X | 0.229 ± 0.066 | 0.099 ± 0.069 |
| 9X | 0.218 ± 0.056 | 0.099 ± 0.068 |
| 10X | 0.211 ± 0.043 | 0.094 ± 0.069 |

Total Cell-Free Hb Removed: 1.849 g

### RBC Mechanical Quantification

The viscosity of RBCs in unprocessed RBC units and final post wash RBCs (lox diacycle) were measured to be 9.252±1.477 cP, and 3.928±1.766 cP, respectively. A significant change in RBC viscosity was observed due to the initial dilution of the RBC unit to 45% HCT, followed by removal of cell debris, proteins, and smaller molecules. The final washed RBC concentrate viscosity was higher than fresh RBCs (2.9 cP at 160 s⁻¹ and 37° C.) and is indicative of the advanced age of the RBC units used in this current example.[18] This viscosity is, however, a significant improvement from the aged RBC unit's initial viscosity of 9.252 cP.

At low shear rates, blood behaves as a Casson fluid and is shear thinning, whereas at shear rates above 100 si, it behaves as a Newtonian fluid.[22] The following equation was used to calculate the shear stress on the inner wall of the TFF hollow fiber lumen with the assumption that the RBC suspension behaves as a Newtonian fluid above a shear rate of 100 s⁻¹, and does not require additional analysis based on the Casson fluid model. In equation (4), Pₒ is the pressure at the inlet and P_{L} is pressure at the outlet of an individual hollow fiber in the TFF cartridge. R is the inner radius of the hollow fiber and L is the effective length of each hollow fiber.

$${τ}_{w} = \frac{( {P}_{o} - {P}_{L} ) R}{2 L} \qquad ( 4 )$$

The shear rate value was extrapolated to 3670 s⁻¹ from manufacturer provided values of 4000 s⁻¹ at a flow rate of 1.09 L/min. The pressure drop within the TFF system from the inlet to the outlet of the hollow fiber cartridge was measured at an average value of 2 psig over 10 diacycles. From this value, we calculated the shear stress to be 12.9 Pa, which is not significantly higher than physiological conditions, and significantly lower than hemolytic shear stress levels of ˜400 Pa.[23,24] By exposing the aged RBC unit to significant shear stress prior to transfusion, RBCs with weakened cell membranes are lysed and removed from the system. From the applied shear stress, we obtained the theoretical viscosity of 3.5 cP for the washed RBC suspension, which corroborates the experimentally measured viscosity using rheometry.

### Oxygen Equilibrium Measurements

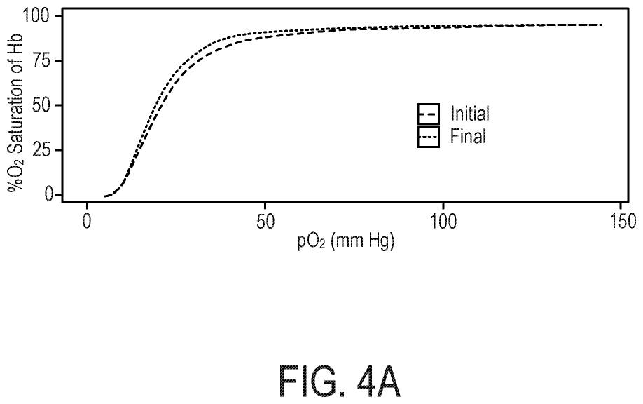

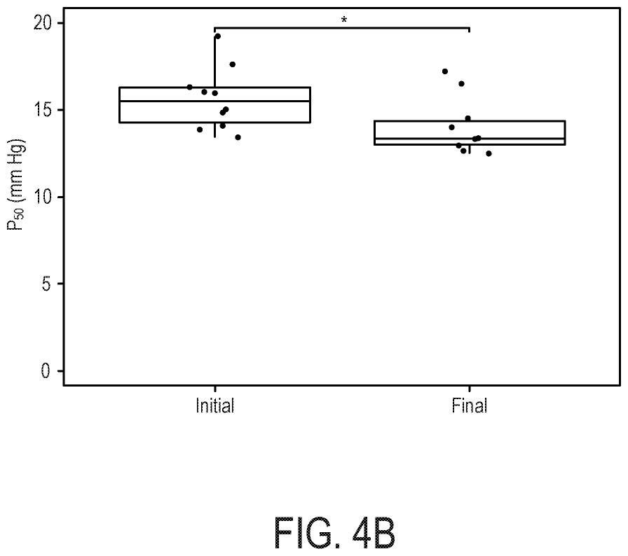

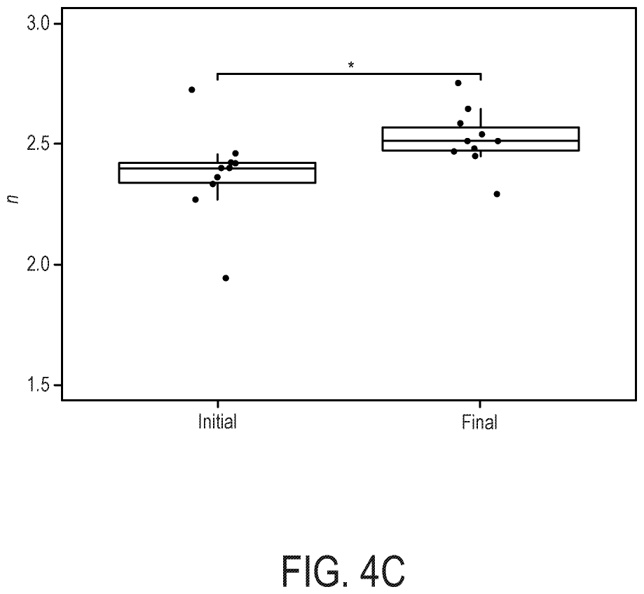

To confirm that TFF-facilitated RBC washing does not negatively affect the oxygen delivery characteristics of RBCs, the oxygen equilibrium curve (OEC) of RBCs pre and post wash were measured. The OEC of RBCs directly from the unprocessed RBC unit (initial) and after the 10× diacycle (final) is shown in FIG. 4A. The left shift of the curve after washing is indicative of the higher oxygen affinity of washed RBCs versus unwashed RBCs.

The OEC provides key details about the ability of the Hb encapsulated in the RBC to bind and release oxygen, which is represented by the regressed P₅₀ and n. A direct comparison between P₅₀ values of the initial unwashed RBCs and the final washed RBCs shows that the P₅₀ decreased from an initial value of 15.6±1.8 mm Hg to 14±1.62 mm Hg post wash (p=0.0493, *) (FIG. 4B). The Hill coefficient (n) comparison between the initial unwashed RBCs and final washed RBCs shows an increase (p=0.0497, *) from 2.37±0.19 to 2.52 t 0.12 (FIG. 4C). 2,3-bisphosphoglycerate, the allosteric effector that decreases Hb binding affinity to oxygen by stabilizing the tense quaternary state of Hb is significantly depleted during ex vivo RBC storage, which shifts the oxygen equilibrium curve to the left, increasing the oxygen affinity of the RBCs.[25] A left shift in the OEC suggests increased oxygen affinity and tighter binding of oxygen by Hb.[26]

### DISCUSSION

In conclusion, a novel RBC washing technique utilizing TFF for removing hemolysis byproducts in a single RBC unit was proposed, with the primary goal of this example to determine the effectiveness of the system in removing extracellular cell-free Hb without inducing further cellular damage. Quantification and characterization of pre and post wash RBC units was centered around the presence of cell-free Hb due to equipment availability at The Ohio State University and does not include the full extent of analytical methods or analytes that could be used to characterize RBC washing effectiveness. While there was no direct comparison between TFF and manual or automated RBC washing systems, comparisons could be drawn between the results from this example to results in the literature. A recent review of RBC washing technology focusing on manual washing and open and closed automated washing systems compares the removal of immunogenic components within RBC units and the resulting long-term storage impacts.[27] Compared to manual and automatic systems, the present example uses more wash solution volume, but shows improved removal of Hb and comparable RBC recovery. The wash time is longer for the TFF process in part due to the increased wash volume. This example focused on an immediate comparison between the RBC unit pre and post wash and aimed to only verify that the TFF process can remove the majority of cell-free Hb from expired RBC units. The designed system has the potential to revolutionize RBC washing systems in part due to its small physical footprint and sterilizable components. This allows the system to easily be transported for potential bedside ex vivo RBC washing and with the implementation of sterile conditions, could allow for direct transfusion of the post-wash RBCs to mitigate increases in potentially immunogenic byproducts derived from ex vivo storage.

### Example 2

The following example includes data from washing unexpired human RBC units. Unexpired RBC units with an ex vivo age of 30 to 42 days old were processed and analyzed using the same protocols performed on the expired RBC units that are described in Example 1. The data trends observed washing unexpired RBC units are very similar to those observed with expired RBC units.

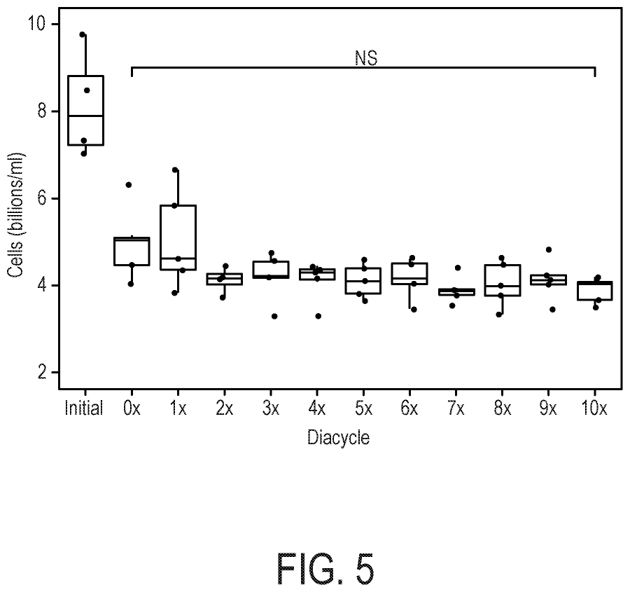

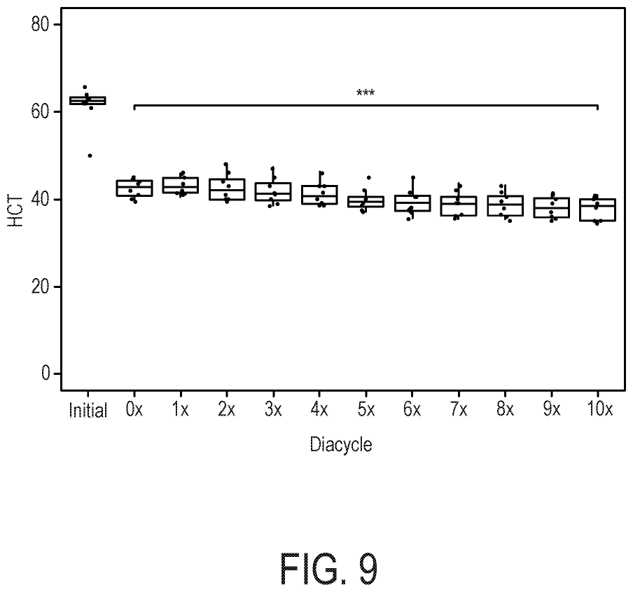

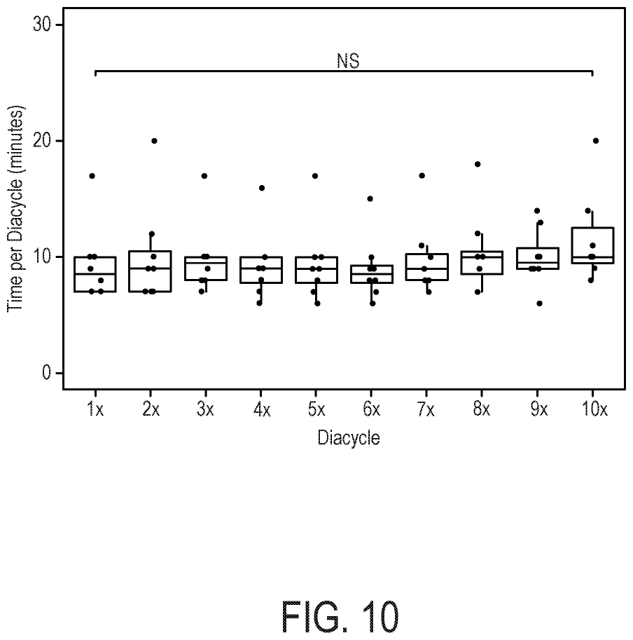

Compared to expired RBC units, there was no significant change in RBC concentration between the 0× and 10× diacycles (p=0.46, NS) in FIG. 5. Contrary to this observation, however, the HCT did decrease significantly during the washing process using unexpired RBCs (p=0.0001333, ***), shown in FIG. 9. The difference was found to be between the first two diacycles and the last two diacycles analyzed using a TukeyHSD test. This contrasts with the cell concentration and HCT data for expired RBC units. Without wishing to be bound by theory, since the unexpired units used were fresher than the expired units, it is possible that the fresher RBC units had a higher mean corpuscular volume that decreased over time as the washing process proceeded and cells lysed, which would drive down the overall HCT, while maintaining similar cell counts. No difference was found comparing the time it takes to complete each diacycle between the unexpired and expired RBC units during the washing process.

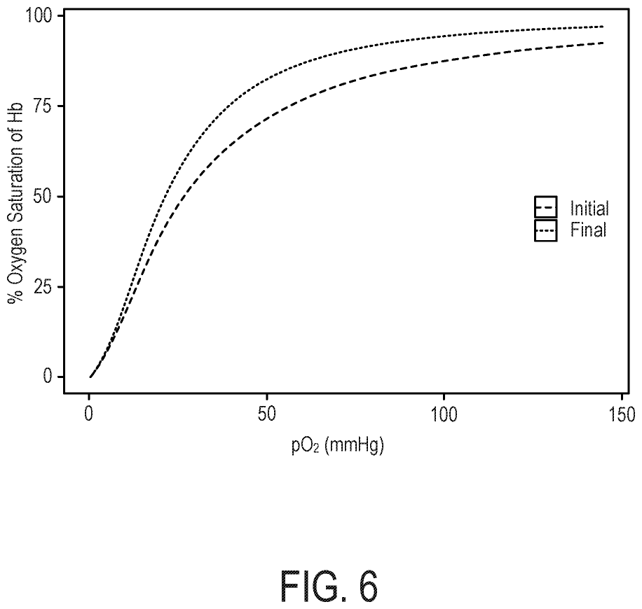

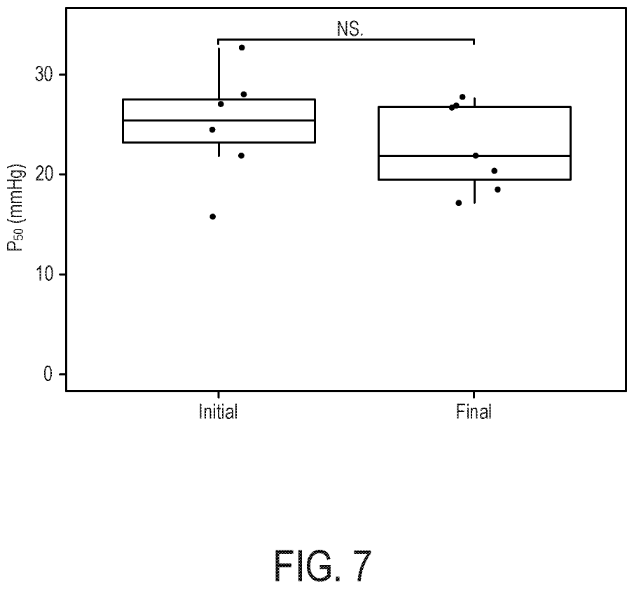

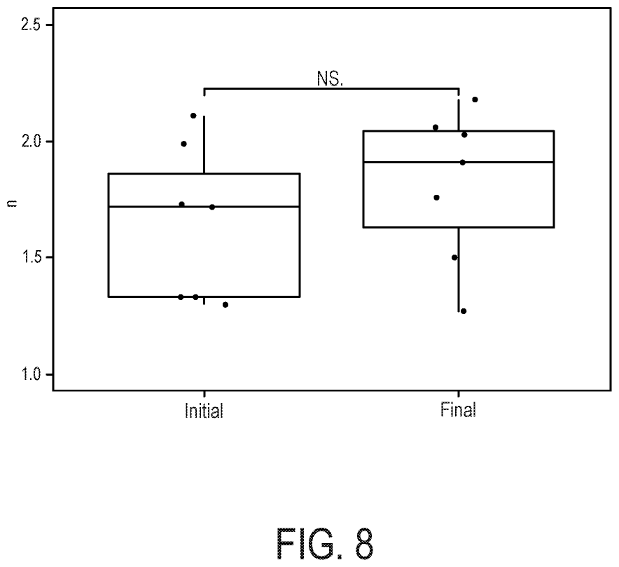

The oxygen equilibrium curve shown in FIG. 6 is similar in shape between the unexpired and expired RBC units. Unexpired RBC units had a higher P₅₀ value (FIG. 7) and lower Hill coefficient (FIG. 8) compared to expired units, and did not show a significant change in either P₅₀ or Hill coefficient from washing by comparing the initial 0× and final 10× diacycles.

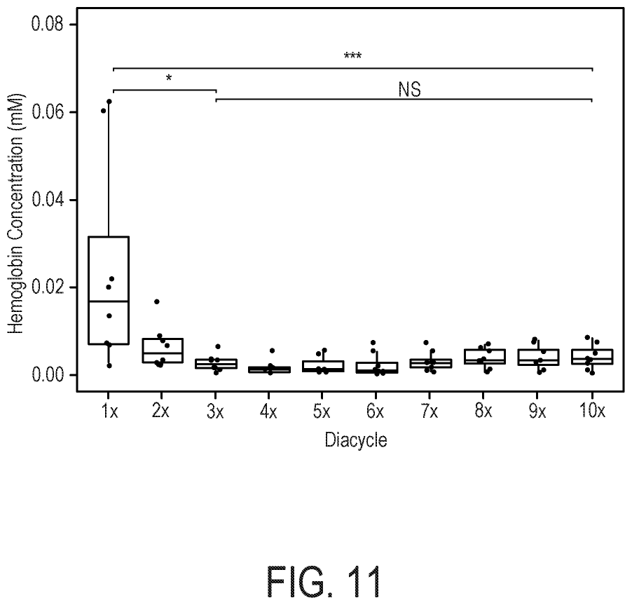

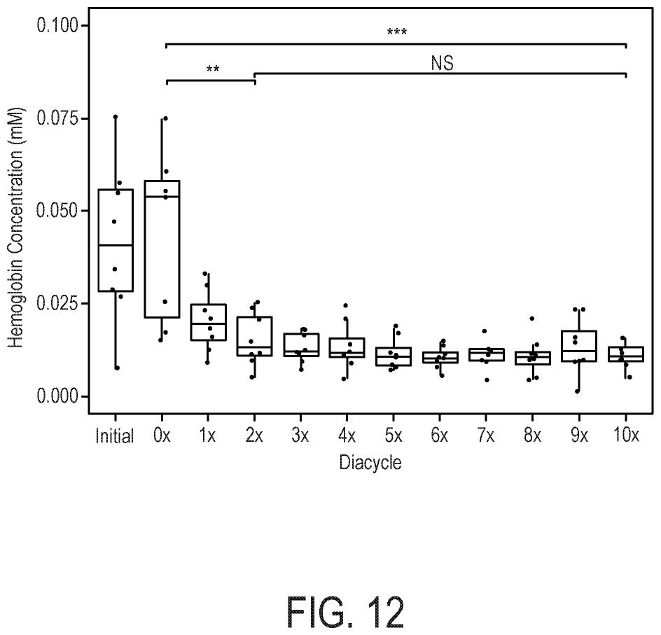

Cell-free Hb concentration both in the permeate and the retentate shows the same trends between washing expired and unexpired RBC units shown in FIGS. 11 and 12. A majority of the cell-free Hb is removed in the first few diacycles and remains constant in the later diacycles. It should be noted that the cell-free Hb concentration is significantly lower for unexpired RBC units compared to expired RBC units, but the Hb concentration appears to increase during the later diacycles 8× to 10×. The Hb concentration increase in the later diacycles was not found to be statistically significant.

### REFERENCES

1. Food and Drug Administration (FDA). CFR—Code of Federal Regulations Title 21. Vol. 21, Www.Fda.Gov. 2019.
2. Hess J R, Sparrow R L, Van Der Meer P F, Acker J P, Cardigan R A, Devine D V. Red blood cell hemolysis during blood bank storage: Using national quality management data to answer basic scientific questions. Transfusion. 2009; 49(12):2599-603.
3. Buehler P W, Alayash A I. Toxicities of hemoglobin solutions: In search of in-vitro and in-vivo model systems. Transfusion. 2004; 44(10):1516-30.
4. Smith A, McCulloh R J. Hemopexin and haptoglobin: Allies against heme toxicity from hemoglobin not contenders [Internet]. Vol. 6, Frontiers in Physiology. Frontiers Media S.A.; 2015 [cited 2021 Jun. 2]. p. 187. Available from: /pmc/articles/PMC4485156/
5. Cholette J M, Henrichs K F, Alfieris G M, Powers K S, Phipps R, Spinelli S L, et al. Washing red blood cells and platelets transfused in cardiac surgery reduces postoperative inflammation and number of transfusions. Pediatr Crit Care Med. 2012 May; 13(3):290-9.
6. Stapley R, Rodriguez C, Oh J Y, Honavar J, Brandon A, Wagener B M, et al. Red blood cell washing, nitrite therapy, and antiheme therapies prevent stored red blood cell toxicity after trauma-hemorrhage. Free Radic Biol Med. 2015; 85:207-18.
7. Hansen A, Yi Q L, Acker J P. Quality of red blood cells washed using the ACP 215 cell processor: Assessment of optimal pre- and postwash storage times and conditions. Transfusion. 2013; 53(8):1772-9.
8. Bennett-Guerrero E, Kirby B S, Zhu H, Herman A E, Bandarenko N, McMahon T J. Randomized study of washing 40- to 42-day-stored red blood cells. Transfusion. 2014; 54(10).2544-52.
9. Lu M, Lezzar D L, Vöro\ddot{s} E, Shevkoplyas S S. Traditional and emerging technologies for washing and volume reducing blood products. J Blood Med [Internet]. 2019 [cited 2021 Jun. 2]; 10:37-46. Available from: /pmc/articles/PMC6322496/
10. O'Leary M F, Szklarski P, Klein T M, Young P P. Hemolysis of red blood cells after cell washing with different automated technologies: Clinical implications in a neonatal cardiac surgery population. Transfusion. 2011, 51(5):955-60.
11. Bennett-Guerrero E, Kirby B S, Zhu H, Herman A E, Bandarenko N, McMahon T J. Randomized study of washing 40- to 42-day-stored red blood cells. Transfusion [Internet]. 2014 Oct. 1 [cited 2021 Nov. 8]; 54(10):2544-52. Available from: https://onlinelibrary.wiley.com/doi/full/10.1111/trf.12660
12. Johnson L, Kwok M, Marks D C. Preparation of red blood cell concentrates and plasma units from whole blood held overnight using a hollow-fibre separation system. Transfus Med. 2015; 25(1):13-9.
13. Brune T, Hannemann-Pohl K, Nißle K, Ecker N, Garritsen H. Quality, stability, and safety data of packed red cells and plasma processed by gravity separation using a new fully integrated hollow-fibre filter device. Adv Hematol. 2009; 2009.
14. Castino F, Wickramasinghe S R. Washing frozen red blood cell concentrates using hollow fibres. J Memb Sci. 1996; 110(2):169-80.
15. Winterbourn C C. Oxidative reactions of hemoglobin. Methods Enzymol. 1990; 186(C):265-72.
16. Hill A V. The possible effects of the aggregation of the molecules of hemoglobin on its dissociation curves. J Physiol. 1910 January; 40:i-vii.
17. Cabrales P, Martini J, Intaglietta M, Tsai A G. Blood viscosity maintains microvascular conditions during normovolemic anemia independent of blood oxygen-carrying capacity. Am J Physiol—Hear Circ Physiol. 2006; 291(2):581-90.
18. Cabrales P, Tsai A G, Intaglietta M. Is resuscitation from hemorrhagic shock limited by blood oxygen-carrying capacity or blood viscosity?Shock. 2007; 27(4):380-9.
19. Yoon C, Noh S, Lee J C, Ko S H, Ahn W, Kim H C. Influence of the washing program on the blood processing performance of a continuous autotransfusion device. J Artif Organs. 2014; 17(1):118-22.
20. Ni J, Zhu W, Wang Y, Wei X, Li J, Peng L, et al. A Reference chart for clinical biochemical tests of hemolyzed serum samples. J Clin Lab Anal. 2021; 35(1):1-7.
21. Sowemimo-Coker S O. Red blood cell hemolysis during processing. Transfus Med Rev. 2002 January; 16(1):46-60.
22. Fournier R L. Basic transport phenomena in biomedical engineering. 2nd ed. New York: Taylor & Francis; 2007. 450 p.
23. Koutsiaris A G, Tachmitzi S V., Batis N. Wall shear stress quantification in the human conjunctival pre-capillary arterioles in vivo. Microvasc Res. 2013; 85(1):34-9.
24. Paul R, Apel J, Klaus S, Schugner F, Schwindke P, Reul H. Shear stress related blood damage in laminar Couette flow. Artif Organs. 2003; 27(6):517-29.
25. Valtis D J, Kennedy A C. DEFECTIVE GAS-TRANSPORT FUNCTION OF STORED RED BLOOD-CELLS. Lancet. 1954 January; 263(6803):119-25.
26. Hamilton C, Steinlechner B, Gruber E, Simon P, Wollenek G. The oxygen dissociation curve: Quantifying the shift. Perfusion. 2004; 19(3):141-4.
27. Cardigan R, New H V., Tinegate H, Thomas S. Washed red cells: theory and practice. Vox Sang. 2020 Nov. 1; 115(8):606-16.

1. A method for removing a contaminate from a blood product, the method comprising:
filtering the blood product by filtration against a filtration membrane using a low-shear pump, thereby forming a retentate fraction comprising a washed blood product and a permeate fraction comprising the contaminate.

2. The method of claim 1, wherein the filtration membrane has a filter rated for removing solutes having a molecular weight or molecular diameter of from 1 g/mol to 30 μm, such as from 1 g/mol to 25 μm, from 1 g/mol to 20 μm, from 1 g/mol to 15 μm, from 1 g/mol to 10 μm, from 1 g/mol to 5 μm, from 1 g/mol to 4 μm, from 1 g/mol to 3 μm, from 1 g/mol to 2 μm, from 1 g/mol to 1 μm, from 1 g/mol to 0.65 μm, from 1 g/mol to 0.2 μm, from 1 g/mol to 0.1 μm, from 1 g/mol to 50 nm, from 1 g/mol to 750 kDa, from 1 g/mol to 500 kDa, from 1 g/mol to 300 kDa, from 1 g/mol to 100 kDa, from 1 g/mol to 70 kDa, from 1 g/mol to 50 kDa, from 1 g/mol to 30 kDa, from 1 g/mol to 10 kDa, from 1 g/mol to 5 kDa, from 1 g/mol to 3 kDa, or from 1 g/mol to 1 kDa.

3. The method of claim 1, wherein the contaminate comprises an extracellular protein, such as hemoglobin (Hb), extracellular vesicles, cytokines, heme, iron, potassium ions, lactate, protons, or any combination thereof.

4. (canceled)

5. The method of claim 1, wherein the inner wall of the filtration membrane in contact with the blood product is subjected to a shear rate less than 5,000 s⁻¹, such as less than 4,000 s⁻¹, less than 3,000 s⁻¹, less than 2,000 s⁻¹, less than 1,000 s⁻¹, or less than 500 s⁻¹.

6. The method of claim 1, wherein the low-shear pump comprises a centrifugal pump.

7. The method of claim 1, wherein the blood product comprises whole blood, packed red blood cells, platelets, white blood cells, or plasma.

8. (canceled)

9. The method of claim 1, wherein the blood product is filtered at a temperature less than 37° C., such as less than 30° C., less than 25° C., less than 20° C., less than 15° C., less than 10° C., less than 5° C., less than 0° C.

10. The method of claim 1, wherein the contaminate is present at a first concentration in the blood product and present at a second concentration in the washed blood product; and
wherein the second concentration is lower than the first concentration.

11. The method of claim 10, wherein the second concentration is 50% or less of the first concentration, such as 40% or less of the first concentration, 30% or less of the first concentration 20% or less of the first concentration, 10% or less of the first concentration, 5% or less of the first concentration, 2.5% or less of the first concentration, or 1% or less of the first concentration.

12. The method of claim 1, wherein filtering the blood product comprises washing the blood product by filtration against a filtration membrane using a low-shear pump.

13. The method of claim 1, wherein filtering the blood product by filtration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles.

14. The method of claim 10, wherein the second concentration is 50% or less of the first concentration after the blood product is filtered for 4 diafiltration cycles.

15. The method of claim 1, wherein the filtration comprises tangential flow filtration.

16. The method of claim 1, wherein the filtration comprises constant-volume filtration.

17. The method of claim 1, wherein the blood product comprises an expired blood product or an unexpired blood product.

18. (canceled)

19. The method of claim 17, wherein the expired blood product comprises a blood product that is at least 45 days from the date of collection, such as at least 50 days from the date of collection, at least 55 days from the date of collection, at least 60 days from the date of collection, at least 65 days from the date of collection, at least 70 days from the date of collection, at least 75 days from the date of collection, at least 80 days from the date of collection, at least 85 days from the date of collection, or at least 90 days from the date of collection.

20. The method of claim 10, wherein the second concentration is 0.05 mM or less, such as 0.04 mM or less, 0.03 mM or less, 0.02 mM or less, or 0.01 mM or less of the contaminate.

21. A red blood cell unit whole blood unit, platelet unit, white blood cell unit, or plasma unit obtained from the method of claim 1.

22-25. (canceled)

26. A system for removing a contaminate from a blood product, the system comprising:
a blood product reservoir for receiving the blood product; and
a filtration unit in fluid communication with the blood product reservoir, the filtration unit comprising
a filtration membrane;
a conduit defining a path for recirculating fluid flow from the blood product reservoir to the filtration membrane and back to the blood product reservoir; and
a low-shear pump operatively coupled to the fluid flow path of the blood product reservoir so as to direct the blood product along the path for recirculating fluid flow.

27-35. (canceled)

36. A method for preparing a regenerated blood product from an expired blood product, the method comprising
filtering the expired blood product by filtration against a filtration membrane using a low-shear pump, thereby forming a retentate fraction comprising the regenerated blood product and a permeate fraction comprising a contaminate.

37-41. (canceled)
